(19) United States
(12) Patent Application Publication
(10) Pub. No.: US 2011/0137083 A1
(43) Pub. Date: Jun. 9, 2011

(54) CONTINUOUS METHOD AND REACTOR FOR HYDROGENATING ORGANIC COMPOUNDS

(75) Inventor: Joachim Pfeffinger, Ludwigshafen (DE)

(73) Assignee: BASF SE, Ludwigshafen (DE)

(21) Appl. No.: 12/994,575

(22) PCT Filed: May 20, 2009
(86) PCT No.: PCT/EP09/56120

§ 371 Date: Nov. 24, 2010

(30) Foreign Application Priority Data
May 27, 2008 (EP) 08156991.5

(51) Int. Cl. C07C 211/35; B01J 19/26

(52) U.S. Cl. 564/450; 422/224

(57) ABSTRACT

The present invention relates to a continuous process for hydrogenating organic compounds in a polyphasic system in the presence of a homogeneous or heterogeneous catalyst, which comprises performing the process in two stages, the first stage being performed in a loop reactor with an external heat exchanger and the second stage in a bubble column reactor with limited backmixing.

The present invention further relates to a hydrogenation reactor (1) in a tall cylindrical design comprising a concentric guide tube (2) arranged in the lower reaction region and a mixing nozzle (3) directed upward, through which reactants and reaction mixture can be supplied, a pump (4) and a heat exchanger (5), which are present in an external pump circulation system which leads from the reactor to the mixing pump, and with one or more gas- and liquid-permeable internals (10) mounted in the upper region of the reactor, where the ratio of total length to diameter of the hydrogenation reactor (1) is in the range from 5:1 to 100:1 and the ratio of the volume of the upper region of the reactor to the volume of the lower region of the reactor is from 0.05:1 to 10:1.

The present invention relates to a continuous process for hydrogenating organic compounds in a polyphasic system comprising dissolved or suspended catalyst, which comprises performing the process in two stages, the first stage being performed in a loop reactor with an external heat exchanger and the second stage in a bubble column reactor with limited backmixing. The present invention further relates to a reactor in which an inventive two-stage hydrogenation can be carried out.

Catalytic hydrogenations of organic compounds are generally performed over heterogeneous catalysts, the catalyst frequently being arranged as a fixed bed. The hydrogenation of unsaturated compounds generally releases considerable amounts of heat, as a result of which the fixed bed reactors have to be equipped with in some cases very complex devices for heat removal. If an additional task is to perform the hydrogenation with maximum conversion, a plurality of cooled fixed bed reactors has to be connected in series. In the course of hydrogenations, even after a short operating time, there may be a decline in the activity and/or the selectivity of the catalyst, which necessitates an exchange or, if possible, a regeneration of the catalyst. In a fixed bed reactor, this catalyst exchange or the regeneration generally cannot be performed in continuing operation, and so the shutdown of the hydrogenation plant becomes necessary.

Alternatively, a heterogeneously catalyzed hydrogenation can be performed in the form of a suspension reaction, in which case the hydrogenation catalyst is suspended by supplying mechanical energy, for example in a stirred tank or in a loop reactor; cf., for example, Ullmann's Encyclopedia of Industrial Chemistry, Verlag Chemie, Electronic Edition Release 2008, 7th Edition, chapter “Reactor Types and Their Industrial Application”, page 24, FIG. 11.

The preparation of cycloaliphatic compounds by hydrogenating aromatic compounds with suspended catalyst is described in numerous examples in the literature, such as DE-C1-907294, U.S. Pat. No. 2,606,925, DE-A1-2132547, U.S. Pat. No. 4,394,522, U.S. Pat. No. 4,448,995 or DE-A1-3226889. It is evident from these examples that the temperature in the reaction can influence both the isomer distribution and the formation of by-products.

The literature likewise includes numerous examples of the industrial implementation of hydrogenations.

DE-A1-1793452 describes a continuous process for performing catalytic hydrogenations, in which a portion of liquid reaction medium is drawn off from the reactor, conducted through a cooler and then fed back to the reactor below the liquid surface together with the hydrogen at high speed. The residence time distribution of such a reactor corresponds to the residence time distribution of a fully backmixed stirred tank (“CSTR”).

DE-A1-19647126 describes a multistage bubble column reactor with installed perforated plates for cascading of the reactor.

EP-A2-57 364 discloses a reactor with installed intermediate trays for hydrogenation of carbon at high pressure, the intention being to prevent backmixing in the reactor by virtue of the intermediate trays.

EP-A2-985446 describes a two-stage continuous process for hydrogenating glucose to sorbitol, wherein the first stage is effected in a stirred vessel and the second stage in a bubble column reactor. This combination allows the residence time to be adjusted such that a conversion of the feedstock of up to 99.1% becomes possible.

DE-A1-10119135 describes a continuous process for preparing diaminodicyclohexylmethane in a reactor system with a plurality of suspension reactors connected in series. Both stirred tank reactors and bubble column reactors are described as possible individual reactors.

DE-A1-2039818 discloses a process and a reactor connection for two-stage hydrogenation of diaminodiphenylmethanes. The reaction is performed initially in a circulation reactor with suspended catalyst, the catalyst is removed and recycled into the circulation reactor, and the liquid is fed to a second reactor with a fixed bed catalyst for complete conversion.

The documents cited reveal that the temperature in the hydrogenation of organic compounds, especially in the hydrogenation of aromatic compounds, can influence both the by-product formation and the isomer ratio. When the temperatures are too low, there is generally the risk that the reaction does not start up or proceeds too slowly, while excessively high temperatures can result in undesired side reactions proceeding up to and including the runaway of the reaction.

The conversion in the hydrogenation should additionally be at a maximum, especially in the case of the hydrogenation of aromatic compounds to the corresponding cycloaliphatic compounds, since the subsequent purification of the resulting reaction mixture, for example by distillation, can otherwise be complicated, since unconverted starting materials may form by-products which are difficult to remove and difficult to handle in the workup.

The prior art discloses, firstly, the use of reactors with good mixing for continuous hydrogenation of aromatic compounds, which have a residence time distribution of a continuous stirred tank reactor.

The residence time distribution in such reactors dictates that a high conversion in the hydrogenation in a stirred tank reactor can be achieved only in batchwise operation or, in continuous mode, virtually only at low volume flows. The heat removal in a stirred tank, especially in the case of a large volume, is also only possible to an insufficient degree.

On the other hand, the prior art discloses bubble column reactors as continuous hydrogenation reactors.

The use of bubble column reactors which are provided with internals which restrict backmixing can prolong the residence time and hence increase the conversion in the process. However, the removal of the heat of reaction in bubble column reactors is, similarly to that in stirred tank reactors, limited.

The combination of stirred tank reactors and bubble column reactor allows a high conversion to be achieved in continuous operation. The problem of removing heat in the main reaction part (stirred tank) of the reactor is, however, not solved. In addition, the technical implementation of such processes is very complicated and hence costly.

The series connection of a plurality of bubble column reactors or stirred tank reactors to form a cascade does enable the achievement of higher conversions in continuous operation, but the problem of removing heat in exothermic reactions is not solved by this type of reaction regime either. In addition, the capital costs increase, as do the complexity of the process regime and the industrial complexity in the case of coupling several reactors.

The coupling of a loop reactor and a tubular reactor with a fixed catalyst bed requires the technically complicated and expensive removal of suspended catalyst after the first process stage.

It was therefore an object of the present invention to develop a technically simple process for hydrogenating organic compounds, with which it is also possible to carry out highly exothermic hydrogenations within a narrow temperature range, with a high throughput and with a very high end conversion in continuous mode. The process according to the invention should enable the reaction to be performed within a defined temperature range in order thus to control the isomer ratio of the reactants and to prevent the formation of undesired by-products. The process should enable a high conversion, in order to simplify the removal of unconverted reactants. Furthermore, the reaction regime and the apparatus configuration of the process should be implementable in a technically simple and economically viable manner. In addition, this process should enable long service lives, such that the number of undesired shutdowns of the reactors for exchange or regeneration of the catalyst can be reduced. The object is achieved in accordance with the invention by a continuous process for hydrogenating organic compounds in a polyphasic system in the presence of a homogeneous or heterogeneous catalyst, which comprises performing the process in two stages, the first stage being performed in a loop reactor with an external heat exchanger and the second stage in a bubble column reactor with limited backmixing.

The present invention is suitable for performing hydrogenations in a polyphasic system. In such a polyphasic system, hydrogen constitutes the gas phase, while the organic compound is present in the liquid phase. A catalyst may be dissolved in the liquid phase, or the catalyst may be present as a solid phase in suspension in the liquid phase.

Hydrogen is used in the process according to the invention.

The hydrogen used for hydrogenation is generally used in a relatively large stoichiometric excess of from 1 to 10 times, preferably from 1.1 to 5 times, the amounts needed in stoichiometric terms. It can be recycled into the reaction as cycle gas. The hydrogen is generally used in technical-grade purity. The hydrogen can also be used in the form of a hydrogen-comprising gas, i.e. in admixtures with other inert gases, such as nitrogen, helium, neon, argon or carbon dioxide. The hydrogen-comprising gases used may, for example, be reformer offgases, refinery gases, etc. However, preference is given to using pure hydrogen or essentially pure hydrogen in the process.

In the process according to the invention, it is additionally possible to use organic compounds which comprise at least one unsaturated carbon-carbon, carbon-nitrogen or carbon-oxygen bond.

Examples of such organic compounds are compounds which comprise at least one or more than one carboxamide group, nitrile group, imine group, enamine group, azide group or oxime group, which are hydrogenated to amines.

In addition, it is possible in the process according to the invention compounds which comprise at least one or more than one carboxylic ester group, carboxylic acid group, aldehyde group or keto group, which are hydrogenated to alcohols.

It is also possible in the process to use organic compounds with unsaturated carbon-carbon bonds, such as alkenes and/or alkynes.

Useful organic compounds also include aromatics or heteroaromatics, which may be converted partially or completely to unsaturated or saturated carbo- or heterocycles. The organic compounds used are preferably aromatic compounds.

Useful aromatic starting materials for the hydrogenation by the process according to the invention include: aromatic monoamines such as aniline, the isomeric toluidines, the isomeric xylidines, 1- or 2-aminonaphthalene, benzidine and substituted benzidines;

aromatic diamines such as the isomeric phenylenediamines (o-phenylenediamine, m-phenylenediamine, p-phenylenediamine), the isomeric tolylenediamines such as 2,4-diaminotoluene and/or 2,6 diaminotoluene, the isomeric diaminonaphthalenes such as 1,5-diaminonaphthalene, bis(4-aminophenyl)methane (MDA), meta-xylenediamine (MXDA), bis(4-amino-3-methylphenyl)methane and bis(4-amino-3,5-dimethylphenyl)-methane;

aromatic polyamines such as polymeric MDA (polymethylenepolyphenylamine);

aromatic mono- and dicarboxylic acids and esters thereof, such as benzoic acid, the isomeric phthalic acids and esters thereof, preferably the methyl esters thereof,

aromatic aminocarboxylic acids and esters thereof, such as anthranilic acid;

aromatic alcohols such as phenol and bisphenol A; and

aromatic hydrocarbons such as benzene, toluene, ethylbenzene, the isomeric xylenes, indene, tetralin and naphthalene.

It is also possible in the process to use substituted aromatic compounds in which, as well as the hydrogenation of the aromatic ring, hydrogenation of the substituents may also occur. Examples thereof are the aromatic monoamines which have nitro groups, such as the isomeric nitroanilines, or aromatic ketones such as acetophenone, or substituted nitriles such as benzonitrile, tolunitrile or o-aminobenzonitrile.

In addition, it is possible to use aromatic nitrogen-containing heterocycles such as pyridine, pyrrole or indole in the process according to the invention.

Preference is given to using, in the process, aromatic amines such as the aforementioned aromatic mono-, di- and/or polyamines.

Particular preference is given to using polymeric MDA, aniline, 2,4-diaminotoluene, 2,6-diaminotoluene, o-phenylenediamine, m-phenylenediamine, p-phenylenediamine, bis(4-aminophenyl)methane (MDA), meta-xylenediamine (MXDA), bis(4-amino-3-methylphenyl)methane and/or bis(4-amino-3,5-dimethylphenyl)methane in the process. Very particular preference is given to using aniline, meta-xylenediamine (MXDA), bis-(4-aminophenyl)methane (MDA), bis(4-amino-3-methylphenyl)methane and bis(4-amino-3,5-dimethylphenyl)methane in the process.

The aromatic amines may, in addition to the amino groups, have no further substituents, or they may bear one or more further substituents, for example alkyl, cycloalkyl, aryl, heteroaryl, halogen, haloalkyl, silyl, hydroxyl, alkoxy, aryloxy, carboxyl or alkoxycarbonyl substituents.

The cycloaliphatic amines obtainable by the process according to the invention can be used as a synthesis unit for the production of surfactants, medicaments and crop protection compositions, stabilizers including light stabilizers, polymers, polyamides, isocyanates, hardeners for epoxy resins, catalysts for polyurethanes, intermediates for preparing quaternary ammonium compounds, plasticizers, corrosion inhibitors, synthetic resins, ion exchangers, textile assistants, dyes, vulcanization accelerants, emulsifiers and/or as starting substances for the preparation of ureas and polyureas. In particular, it is possible to use cyclohexylamine obtainable by the hydrogenation of aniline as a corrosion inhibitor or vulcanization accelerant.

The hydrogenation products of bis(4-aminophenyl)methane (MDA), bis(4-amino-3-methylphenyl)methane and/or bis(4-amino-3,5-dimethylphenyl)methane can be used as a monomer unit for polyamides, as a hardener for epoxy resins or as a starting material for the preparation of the corresponding isocyanates.

The hydrogenation can be carried out with or without solvent. The use of solvents is advantageous when the organic compound is present in solid form and can be handled and conveyed as a melt only with great complexity, if at all. The solvents used may be alcohols such as isopropanol, isobutanol or t-butanol, or ethers such as diethyl ether, glycol dimethyl ether, dioxane or tetrahydrofuran.

However, the solvent used may also be the end product formed in the reaction.

Useful solvents may also include mixtures of the aforementioned solvents.

Preferred solvents are isopropanol, isobutanol and/or t-butanol. Particular preference is given to using the end product formed in the reaction as the solvent.

The solvent is usually used in such an amount that from 10 to 50% (% by weight), preferably from 15 to 40% and more preferably from 20 to 30% solutions of the organic compounds intended for the hydrogenation are obtained. Particular preference is given to performing the process without using a solvent.

The process according to the invention proceeds in the presence of a homogeneous or heterogeneous catalyst which is suitable for hydrogenation. Preference is given to performing the hydrogenation in the presence of a heterogeneous catalyst.

Useful homogeneous catalysts include liquid and/or soluble hydrogenation catalysts, for example Wilkinson catalysts, Crabtree catalysts or Lindlar catalysts.

The heterogeneous catalysts used are, for example, noble metals such as platinum, palladium, ruthenium and rhodium, or other transition metals such as molybdenum, tungsten, chromium, but particularly iron, cobalt and nickel, either individually or in a mixture.

To increase the activity and stability, it is possible to use heterogeneous catalysts in finely distributed form in the process.

The catalysts may comprise support material.

The support materials used are typically carbon such as graphite, carbon black and/or activated carbon, aluminum oxide, silicon dioxide, zirconium dioxide, zeolites and aluminosilicates, and also mixtures of these support materials.

The shape and size of the support material may vary. Typical mean particle sizes of support materials are in the range from 0.0001 up to 5 millimeters, preferably from 0.0005 to 1 mm, more preferably from 0.001 to 0.5 mm.

The concentration of the catalyst is typically 1% by weight and less, preferably 0.1% by weight and less, and more preferably 0.01% by weight and less, based on the sum of the mass of the organic compound used and the mass of the solvent used.

In a particular embodiment, the heterogeneous Ru-containing catalyst used for the hydrogenation of aromatic compounds is preferably a catalyst based on ruthenium oxide hydrate. Particular preference is given to using an Ru-containing catalyst as disclosed in DE-A1-2132547. The preparation of such preferred catalysts is described in detail, for example, on pages 4 to 5 and in Example 1 of DE-A1-2132547. Typically, the mean particle size of heterogeneous Ru-containing catalysts which have been obtained by precipitation is in the range from 1 nm to 1 μm, preferably in the range from 3 nm to 100 nm. For example, the mean particle size of the ruthenium particles which are used in the form of oxide hydrates of Ru, according to the disclosure of DE-A1-2132547 is between 4 and 6 nm.

The heterogeneous catalysts are typically used in the form of a suspension of the catalyst in the liquid reactants or solvents used.

The process according to the invention is performed typically at a pressure of from 1 to 500 bar, preferably at a pressure of from 50 to 325 bar, more preferably a pressure of from 150 to 250 bar.

The process is generally performed at a temperature in the range of 50 and 300° C., preference being given to the temperature range from 120 to 280° C.

The process according to the invention is performed in two stages.

The first stage is performed in a loop reactor with external heat exchanger.

Examples of loop reactors are tubular reactors with internal and external loops. Such reactors are described in detail, for example, in Ullmann's Encyclopedia (Ullmann's Encyclopedia of Industrial Chemistry, Verlag Chemie, Electronic Release 2008, Th Edition, chapter “Stirred Tank and Loop Reactors” and chapter “Bubble Columns”).

A loop reactor consists typically of a vertical, preferably cylindrical, tubular reactor. The ratio of length to diameter of the loop reactor is typically from 2:1 to 100:1, preferably from 5:1 to 100:1, more preferably from 5:1 to 50:1, especially preferably from 5:1 to 30:1.

The reactants are fed to the tubular reactor, individually or mixed.

The reactants are fed in preferably in the lower region of the tubular reactor, preferably through a mixing nozzle disposed in the lower region of the loop reactor.

The term “mixing nozzle” refers, in a customary manner, to a tube which narrows in flow direction.

Hydrogen gas, reactants which may be dissolved in a solvent, and catalyst solution or catalyst suspension are generally mixed intensively in the mixing nozzle and fed to the reactor.

The mixing nozzle may be designed as a one-substance or two-substance nozzle.

In the one-substance nozzle, only the liquid reaction mixture is sprayed in and the hydrogen is fed to the reactor at any other position, but preferably in the liquid phase, for example by means of a sparging or distributor ring. What is advantageous about this configuration is the simple construction of such a one-substance nozzle; what is disadvantageous is the relatively poor dispersion of the hydrogen in the reaction mixture.

In the two-substance nozzle, the hydrogen is fed in and dispersed together with the liquid reaction mixture. In this embodiment, a high dispersion of the hydrogen in the reaction mixture is typically achieved.

The mixing nozzle is preferably configured as a two-substance nozzle.

In the context of the present application, two-substance nozzle also refers to a nozzle in which catalyst particles are present suspended in the liquid reaction mixture.

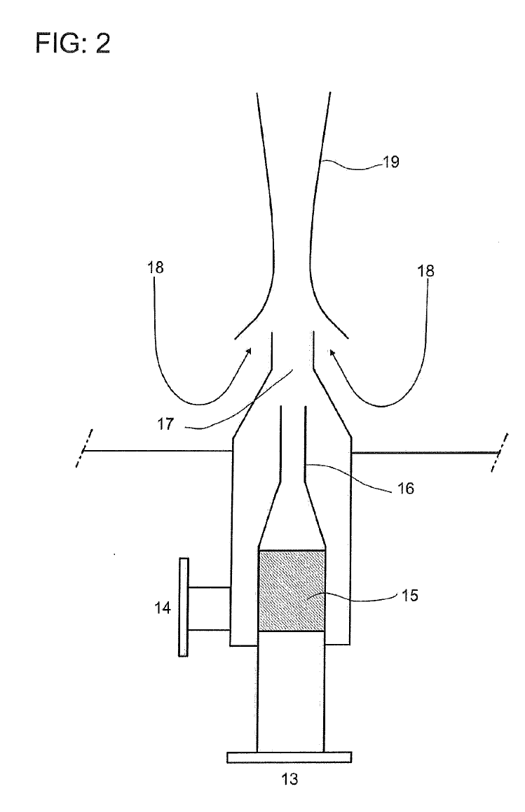

A preferred embodiment of the mixing nozzle is shown in FIG. 2 and is described in detail below.

The loop reactor is generally configured as a tubular reactor with an external circulation system (external loops).

In a loop reactor with an external circulation system, a draw line is generally present at any desired position in the reactor, preferably in the lower region of the reactor, through which the reaction mixture is conducted within an outer circulation system by means of a conveying unit back to the mixing nozzle.

The conveying unit is preferably a pump, and the external circulation system is therefore typically referred to as a pumped circulation system.

Examples of pumps are centrifugal pumps or rotary piston pumps including rotary vane pumps or gear pumps. Particular preference is given to using centrifugal pumps as the conveying unit.

In the process according to the invention, the first stage is performed in a loop reactor with external circulation, in which case a heat exchanger is present in the external circulation system. In the context of this invention, such a reactor is referred to as a loop reactor with an external heat exchanger.

The heat exchanger is, for example, a tube bundle heat exchanger, double tube heat exchanger, plate heat exchanger or spiral heat exchanger. At design pressures for the reactor below 100 bar, preference is given to using a tube bundle heat exchanger; at higher pressures, preference is given to using one or more series-connected double tube heat exchangers.

The loop reactor with an external heat exchanger is typically operated in such a way that a portion of the reaction mixture is conveyed out of the reactor through the external pumped circulation system in which the external heat exchanger is present, which cools the reaction mixture conveyed through the heat exchanger.

The external pumped circulation generally generates so-called external loop flow. As a result, the reaction mixture in the first reaction stage is generally mixed and circulated to such a high degree that the residence time in the first stage typically corresponds to that of a fully backmixed stirred tank (CSTR).

The reaction mixture is finally fed back to the reactor by means of a mixing nozzle. Typically, fresh reactants, hydrogen gas and catalyst solution or catalyst suspension are metered into the pumped circulation system and fed to the reactor as a reaction mixture together with the stream already present in the pumped circulation system, preferably by means of a mixing nozzle. Hydrogen gas is generally also supplied to the mixing nozzle, said hydrogen gas being removed from the reaction mixture in a gas separator downstream of the first and/or second reaction stage (cycle gas).

In a preferred embodiment, the loop reactor is configured such that, in addition to the external loop flow, a so-called internal loop flow is formed. In a loop reactor with internal loop flow, a concentric, preferably cylindrical guide tube is generally arranged within the interior of the tubular reactor and extends essentially over the entire length of the tubular reactor with the exception of the reactor ends.

The guide tube is generally configured as a simple tube. The ratio of length to diameter of the guide tube is generally from 5:1 to 100:1, preferably from 5:1 to 50:1.

The diameter of the guide tube is typically less than the diameter of the tubular reactor. The ratio of the diameter of the guide tube to the diameter of the tubular reactor is generally from 0.3:1 to 0.9:1, preferably from 0.5:1 to 0.7:1.

The space between the guide tube and the reactor inner wall is generally referred to as the ring space.

The mixing nozzle is typically arranged such that the gas/liquid jet generated by the mixing nozzle is directed into the guide tube.

The mixing nozzle is preferably arranged below the lower end of the guide tube, especially spaced apart by from ⅛ of the guide tube diameter up to one guide tube diameter, or is immersed into the guide tube, in a depth up to several guide tube diameters.

The gas/liquid jet generated by means of the mixing nozzle brings about flow within the guide tube (upflow column), which, after leaving the guide tube, is deflected such that the liquid is returned in the direction of the mixing nozzle within the ring space between guide tube and reactor inner wall (downflow column). This generally gives rise to an internal loop flow. When the mixing nozzle is present at the lower end of the tubular reactor, the internal loop flow is typically enhanced by the bubble column which forms within the guide tube through exploitation of gas buoyancy.

The ratio of the volume flows of internal loop flow to reaction mixture pumped in external circulation is preferably from 2 to 30:1, more preferably from 5 to 20:1.

The loop reactor with internal loop flow may also be configured with two separate pipelines, in which case the upward flow is carried out in a first pipeline, gas and liquid are separated above this pipeline and the liquid is conducted back downward to the mixing nozzle in a second pipeline.

The loop reactor with external pumped circulation with a heat exchanger can also be configured as a stirred tank reactor with external pump circulation with a heat exchanger. In this embodiment, the reactants, as described above, can preferably be fed in by feeding them into a pumped circulation system and introducing the reaction mixture by means of a mixing nozzle. Preference is given to feeding the reactants into the lower part of the reactor. The stirring generally achieves good mixing in the reactor, and the heat of reaction can be removed by means of the external heat exchanger.

However, preference is given to performing the first process stage in a loop reactor with internal loop flow.

A portion of the reaction mixture is typically fed from the first reaction stage to the second reaction stage.

According to the invention, the second reaction stage is a bubble column reactor with restricted backmixing, i.e. the circulation of gas and liquid is restricted such that the residence time distribution in this second reaction stage corresponds to that of a tubular reactor.

This defined liquid residence time allows the conversion of the reactants to proceed virtually to completion. In addition, no further cooling apparatus is generally required in the second reaction stage, since only low heat of reaction is released.

The bubble column reactor consists typically of a vertical, preferably cylindrical, tubular reactor.

The ratio of length to diameter of the bubble column reactor is typically from 2:1 to 100:1, preferably from 5:1 to 50:1, more preferably from 10:1 to 30:1.

The backmixing in the bubble column reactor of the second process stage is preferably restricted by internals. The incorporation of such apparatus generally limits the circulation and hence the backmixing of gas and liquid. The internals are typically such that the polyphasic mixture consisting of gas, liquid and dissolved or suspended catalyst can flow through together, without any separation of the phases or else sedimentation and deposition of solid in this reaction part.

The restriction of backmixing in the bubble column reactor can be implemented, for example, by the incorporation of various internals:

In a preferred embodiment, backmixing is restricted by the incorporation of a plurality of fixed trays in the tubular reactor.

In general, individual segments (“compartments”) with defined reaction volumes thus arise between the individual trays. Each of the individual segments generally acts like a single, backmixed stirred tank reactor. With increasing number of individual segments in succession, the residence time distribution of such a cascade generally approximates to the residence time of a tubular reactor. The trays are preferably configured as liquid-permeable sparging trays. Examples of such sparging trays are simple perforated plates and slotted trays, it being possible for the sheets or trays to be provided with sparging nozzles or with two-substance valves, and specially constructed sparging trays, for example nozzle trays or Thormann trays, tunnel-cap trays or crossflow trays.

The sparging trays are preferably configured as perforated plates, slotted trays, nozzle trays, Thormann trays, tunnel-cap trays or crossflow trays. The sparging trays are more preferably configured as perforated sheets.

The volume of the bubble column reactor is typically divided by the installed trays into individual segments of about equal size.

The number of individual segments thus formed is preferably from 1 to 20, more preferably from 1 to 10, especially preferably from 2 to 6.

The volume of the individual segments is preferably in each case from 5 to 50%, more preferably from 10 to 25%, based on the total volume of the bubble column reactor. However, another distribution of the individual segments is also possible according to the reaction system and heat of reaction released.

In a further preferred embodiment, the backmixing is restricted by the incorporation of irregular beds of random packing or through the incorporation of a structured packing within the entire volume of the bubble column reactor.

In the case of random packings and structured packings, preference is given to selecting a design which brings about further mixing of liquid and gas phase and simultaneously possesses a low liquid holdup in order to prevent the sedimentation of catalyst. The material used should generally therefore have minimum porosity and at the same time have no flow-calmed zones. To prevent solid deposits, the surface should preferably be as smooth as possible, for example in the case of metal or glazed ceramic.

The random packings used may, for example, be Pall® rings or Raschig® rings. The designs of structured packings used may generally be all designs which are otherwise customarily used as internals in distillation columns, for example the Mellapack®, Optiflow®, Mellagrid®, BX®, CYO, Mellacarbon®, Melladur®, Kerapak® types (from Sulzer Chemtech), the Montz-Pak® types from Montz, and Flexipac® (from Koch-Glitsch).

The loop reactor of the first reaction stage and the bubble column reactor of the second reaction stage may be arranged as two spatially separate apparatus, which are connected to one another, for example, via pipelines.

In a preferred embodiment, however, both reaction stages, specifically loop reactor and bubble column reactor, are arranged in one apparatus (hydrogenation reactor).

In this preferred embodiment, the hydrogenation reactor is configured as a long, cylindrical tube (tall cylindrical design).

The ratio of total length to diameter of the tube is preferably in the range from 5:1 to 100:1, more preferably from 5:1 to 50:1, most preferably from 10:1 to 30:1.

The cylindrical tube is typically divided into two regions.

The first region of the hydrogenation reactor is configured as described above as a loop reactor with an external heat exchanger.

The second region of the hydrogenation reactor is configured as described above as a bubble column reactor.

The region of the loop reactor and that of the bubble column reactor are generally separated from one another by internals, preferably sparging trays, such as perforated plates.

The region of the bubble column reactor begins typically from the first installed element which prevents backmixing. In this case, the first incorporated element is also counted within the region of the bubble column reactor.

The region up to the first incorporated element which restricts backmixing is assigned to the region of the loop reactor.

The ratio of the volume of the upper region of the reactor in which the internals are mounted and in which the second stage of the hydrogenation process is carried out to the volume of the lower region of the reactor in which the first stage of the process is carried out is preferably in the range from 0.1:1 to 10:1, more preferably in the range from 0.3:1 to 2:1, most preferably in the range from 0.4:1 to 1.5:1, especially 1:1.

The volume of the upper region of the reactor in which the second process stage is performed is divided by the sparging trays into individual segments (compartments), the volume of one individual segment divided by the sparging trays being preferably in the range from 5 to 50%, based on the total volume of the reactor.

The number of individual segments is preferably from 1 to 20, more preferably from 1 to 10, especially from 2 to 6.

In a very particularly preferred embodiment, the ratio of the volume of the upper region of the reactor in which the internals are mounted and in which the second stage of the hydrogenation process is carried out to the volume of the lower region of the reactor in which the first stage of the process is carried out is in the range from 0.4:1 to 1.5:1, especially 1:1, with a number of from 2 to 6 individual segments. This preferred embodiment enables a sufficient residence time to achieve high conversions, even in the event of conversion and load variations in the first reaction stage.

The process according to the invention, in the preferred embodiment in which loop reactor and bubble column reactor are integrated in one apparatus, is preferably conducted in such a way that a maximum temperature difference of 20 K, preferably less than 10 K, occurs between the reactant feed in the loop reactor of the first reaction stage and the exit of the gas-liquid mixture in the bubble column reactor of the second reaction stage.

The temperature in the loop reactor can be fixed very accurately by the circulation rate and the dimensions of the cooler in the pump circulation system.

The conversion is regulated in this reactor preferably via the amount of catalyst supplied. To achieve a very substantially complete conversion at the exit of the bubble column reactor and to restrict the maximum temperature difference which occurs over the entire reactor to less than 20 K, preferably less than 10 K, it has been found to be advantageous to establish, in the loop reactor of the first process stage, a conversion in the range of 90-98%, preferably from 92 to 98%.

The conversion in the first process stage is preferably regulated by the concentration of the catalyst solution or catalyst suspension supplied. In general, the conversion in the first process stage can be increased by an increase in the catalyst concentration.

The conversion can be monitored by sampling in the pump circulation system or more preferably by means of suitable online analysis methods, preferably by means of infrared or near infrared measurements.

The second reaction stage is generally arranged such that the reaction mixture, by means of the flow generated by the gas buoyancy, passes out of the region which is designed as a loop reactor into the internals of the bubble column reactor.

At the top of the second reaction stage (bubble column reactor) liquid and gas leave the reactor (hydrogenation reactor) together and are generally separated from one another in a downstream separating vessel (gas separator). The separated gas is typically fed back to the mixing nozzle in the first reaction stage (cycle gas).

The loop reactor of the first reaction stage and the bubble column reactor of the second reaction stage may, as mentioned above, also be arranged as two separate apparatuses.

In such an alternative embodiment, the loop reactor, as described above, is configured as a loop reactor with external heat exchanger.

The bubble column reactor is, as described above, configured as a tubular reactor with internals which restrict backmixing.

In a loop reactor with internal loops, an impingement plate may be arranged above the upper end of the guide tube within the upper region of the loop reactor. The impingement plate is generally arranged at right angles to the guide tube and is preferably spaced apart therefrom by from one to two guide tube diameters. The impingement plate is preferably disk-shaped, with a diameter greater than the diameter of the guide tube and less than the internal diameter of the loop reactor. The thickness is generally from about 5 to 10 mm. The impingement plate typically has the task of bringing about a reversal of flow in the upper region of the reactor.

In this alternative process variant, in which the reaction stages are configured as two separate apparatuses, hydrogen gas and the liquid reaction mixture are typically supplied to the loop reactor, preferably by means of a mixing nozzle. At any point in the loop reactor, for example in the lower region or in the pumped circulation system—but upstream of the metered addition of the reactants—a portion of the liquid reaction mixture is branched off and fed to the second reaction stage. The reaction mixture from the first process stage can be fed in by means of a pump, but the feeding can also be effected in such a way that the second process stage is operated at a somewhat lower reaction pressure, and reaction mixture is conveyed into the second process stage as a result of the pressure difference.

The reaction mixture from the first stage is typically metered into the bubble column reactor of the second stage, preferably by means of a nozzle, preferably together with hydrogen gas. However, hydrogen gas can also be supplied separately, for example through a gas introduction or distributor ring, preferably in the lower region of the bubble column reactor. In the lower region of the bubble column reactor, in which the reactants, especially hydrogen gas, are supplied, there are typically no internals. This region in which there are typically no internals corresponds typically to from 5 to 50% of the volume of the bubble column reactor of the second reaction stage. When the internals used are trays such as perforated plates, hydrogen gas can be introduced into the reaction chamber upstream of the first tray or into the reaction space between first and second tray.

The hydrogen gas can be supplied freshly or as cycle gas.

At the top of the second reaction stage, liquid and gas together leave the bubble column reactor and are generally separated from one another in a downstream separating vessel (gas separator). The separated gas is typically fed back to the mixing nozzle in the first and/or second reaction stage (cycle gas).

The reaction is conducted in analogy to the performance of the preferred embodiment of the process, in which loop reactor and bubble column reactor are integrated in one apparatus, in such a way that, between the reactant feed in the loop reactor of the first reaction stage and the exit of the gas-liquid mixture in the bubble column reactor of the second reaction stage, a maximum difference of 20 K, preferably less than 10 K, occurs. The conversion is regulated preferably via the amount of catalyst supplied. To achieve a maximum conversion at the exit from the bubble column reactor and to restrict the maximum temperature difference which occurs over the entire reactor to less than 20 K, preferably less than 10 K, it has been found to be advantageous to establish a conversion in the range of 90-98%, preferably from 92 to 98%, in the loop reactor of the first process stage. The conversion in the first process stage is preferably regulated through the concentration of catalyst solution or catalyst suspension supplied. In general, the conversion in the first process stage can be increased by an increase in the catalyst concentration.

However, the process according to the invention is preferably carried out in an inventive reactor in which the two stages of the process are arranged in one apparatus.

Accordingly, the present invention further provides a hydrogenation reactor (1) in a tall cylindrical design comprising a concentric guide tube (2) arranged in the lower reaction region and a mixing nozzle (3) directed upward, through which reactants and reaction mixture can be supplied, a pump (4) and a heat exchanger (5), which are present in an external pump circulation system which leads from the reactor to the mixing pump, and with one or more gas- and liquid-permeable internals (10) mounted in the upper region of the reactor, where the ratio of total length to diameter of the hydrogenation reactor (1) is in the range from 5:1 to 100:1 and the ratio of the volume of the upper region of the reactor to the volume of the lower region of the reactor is from 0.05:1 to 10:1.

The first stage of the process according to the invention is performed in the lower region of the hydrogenation reactor (1), while the second stage of the hydrogenation process is performed in the upper region of the hydrogenation reactor (1).

In the lower region of the hydrogenation reactor (1) is arranged, in the interior of the tubular reactor, a concentric, cylindrical guide tube (2) which extends essentially over the entire length of the lower region of the tubular reactor up to the internals of the upper region of the reactor, although the internals are spaced apart from the upper end of the guide tube by from one to two guide tube diameters.

The guide tube (2) is generally configured as a simple tube. The ratio of length to diameter of the guide tube is preferably from 5:1 to 100:1, more preferably from 5:1 to 25:1 and most preferably from 10:1 to 20:1.

The ratio of the diameter of the guide tube to the diameter of the tubular reactor is preferably from 0.3:1 to 0.9:1, more preferably from 0.5:1 to 0.7:1.

The space between guide tube and the reactor inner wall is generally referred to as the ring space.

The mixing nozzle (3) is typically arranged such that the gas/liquid jet generated by the mixing nozzle is directed into the guide tube. The mixing nozzle is preferably arranged below the lower end of the guide tube, especially spaced apart by from ⅛ of the guide tube diameter up to one guide tube diameter, or is immersed into the guide tube, in a depth up to several guide tube diameters.

The upper end of the mixing nozzle is preferably immersed into the guide tube by one guide tube diameter.

A draw line is present in the hydrogenation reactor, preferably in the lower region of the reactor, through which the reaction mixture is fed back to the mixing nozzle (3) within an external pumped circulation system by means of a pump (4).

Examples of pumps (4) are centrifugal pumps or rotary piston pumps including rotary vane pumps and gear pumps. Particular preference is given to using centrifugal pumps as the conveying unit.

The term “mixing nozzle” refers, in a customary manner, to a tube which narrows in flow direction. Hydrogen gas, reactants which may be dissolved in a solvent, and catalyst solution or catalyst suspension are mixed intensively in the mixing nozzle and fed to the reactor.

The mixing nozzle can be defined as a one-substance or two-substance nozzle.

In the case of the one-substance nozzle, only the liquid reaction mixture is sprayed in, and the hydrogen is fed to the reactor at any other point, but preferably in the liquid phase, for example by means of a sparging or distributor ring. What is advantageous about this embodiment is the simple construction of such a one-substance nozzle; what is disadvantageous is the relatively poor dispersion of the hydrogen in the reaction mixture.

In the case of the two-substance nozzle, the hydrogen is fed in and dispersed together with the liquid reaction mixture. In this embodiment, a high dispersion of the hydrogen in the reaction mixture is typically achieved.

The mixing nozzle is preferably configured as a two-substance nozzle.

In the context of the present application, a two-substance nozzle also refers to a nozzle in which catalyst particles suspended in the liquid reaction mixture are present. A preferred embodiment of a mixing nozzle is shown in FIG. 2 and is described in detail below.

In the outer circulation system, there is a heat exchanger (5).

Preferably, feed lines (6, 7 and 8) which are provided for the feeding of the reactants are provided in the outer circulation system.

The heat exchanger is, for example, a tube bundle heat exchanger, double tube heat exchanger, plate heat exchanger or spiral heat exchanger. At design pressures for the reactor below 100 bar, preference is given to using a tube bundle heat exchanger; at higher pressures, preference is given to using one or more double tube heat exchangers connected in series.

In the upper region of the hydrogenation reactor are mounted one or more gas- and liquid-permeable internals (10), by means of which the upper region is spatially separated from the lower region of the reactor.

Useful internals include the aforementioned internals, the gas- and liquid-permeable internals (10) preferably being sparging trays, such as perforated plates. The internals may also, as described above, be configured as random packings or structured packings.

The ratio of total length to diameter of the hydrogenation reactor (1) is preferably in the range from 5:1 to 100:1, more preferably from 5:1 to 50:1, most preferably from 10:1 to 30:1.

The design of a hydrogenation reactor as a long, thin tube offers several advantages. In terms of reaction technology, it has been found that a long tube is advantageous since ascending gas bubbles have a comparatively long residence time in the reactor and hence can be converted with a high conversion without having to be recompressed. Viewed in apparatus terms, such a reactor design in particular offers the advantage of simple manufacture from a cylindrical tube. In the case of configuration for high system pressures, moreover, the lower the diameter, the greater the reduction in the wall thickness required.

The ratio of the volume of the upper region of the reactor in which the internals are mounted and in which the second stage of the hydrogenation process is performed to the volume of the lower region of the reactor in which the first stage of the process is performed is preferably in the range from 0.05:1 to 10:1, more preferably in the range from 0.3:1 to 2:1, most preferably in the range from 0.4:1 to 1.5:1, especially 1:1.

The volume of the upper region of the reactor in which the second process stage is performed is divided into individual segments (compartments) by the sparging trays, the volume of an individual segment divided by the sparging trays being preferably in the range from 5 to 50%, based on the total volume of the reactor.

The number of individual segments is preferably from 1 to 20, more preferably from 1 to 10, especially from 2 to 6.

In a very particularly preferred embodiment, the ratio of the volume of the upper region of the reactor in which the internals are mounted and in which the second stage of the hydrogenation process is performed to the volume of the lower region of the reactor in which the first stage of the process is performed is in the range from 0.4:1 to 1.5:1, especially 1:1, with a number of from 2 to 6 segments. This preferred embodiment enables a sufficient residence time to achieve high conversions, even in the event of conversion and load variations in the first reaction stage.

In the upper region of the reactor, there is typically an exit point for the reaction mixture which is separated in a separator (11) into a liquid stream/product stream (12) and a gas stream. The gas stream, which typically comprises hydrogen, can be fed through a cycle gas line (9) to the mixing nozzle (3) or be sent to workup as an offgas stream (20).

The appended drawings show a preferred embodiment of the hydrogenation reactor and a preferred embodiment of the mixing nozzle (3):

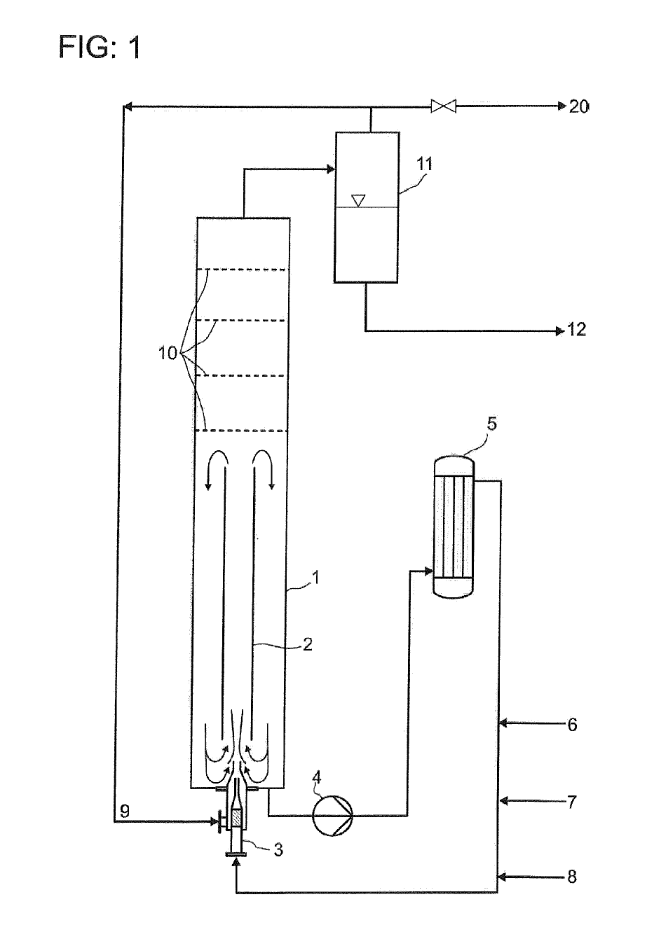

FIG. 1 shows the complete structure of a preferred hydrogenation reactor in which loop reactor and bubble column reactor are integrated in one apparatus, whose structure and mode of function are explained below:

The inventive hydrogenation reactor (1) is designed as a long, cylindrical tube (tall cylindrical design), where, as described, the ratio of total length to diameter is in the range from 5:1 to 100:1, preferably from 5:1 to 50:1, more preferably from 10:1 to 30:1. The reactor is divided by means of a plurality of perforated plates (10), four of them in the example shown, such that the volume in the region of the loop reactor (below the lowermost tray) is approx. 50% and the volumes in the resulting upper chambers (region of the bubble column reactor) are each of equal size, in each case 12.5% in the example shown. However, according to the reaction system and heat of reaction released, another distribution of the individual segments is also possible.

In the lower region of the reactor is disposed a centrally mounted tube, the so-called guide tube (2). In the base of the reactor is centrally mounted a sparging nozzle (3) which projects into the guide tube (2).

Liquid is drawn off from the lower part of the reactor by means of a circulation pump (4) and conducted through a heat exchanger (5) for cooling. Downstream of the heat exchanger, hydrogen gas (6) and liquid reactant (7) and liquid catalyst suspension (8) are added to this circulation liquid. The mixture obtained is finally fed back to the reactor through the mixing nozzle (3). In the mixing nozzle (3), cycle gas (9) is sucked in, compressed and dispersed in the liquid.

In the upper part of the mixing nozzle (3), liquid is sucked in from the ring space between the guide tube (2) and outer jacket of the reactor (1) and mixed further with the liquid-gas mixture.

Above the guide tube (2), liquid and gas separate. The main proportion of the liquid flows back downward outside the guide tube; the excess proportion of the liquid passes together with the gas upward through the perforated plates (10) into the individual segments of the bubble column reactor.

At the top of the reactor, the liquid-gas mixture leaves and is separated in the separating vessel (11) into a liquid phase and a gaseous phase. The gaseous phase is fed completely or partially to the mixing nozzle (3) via a cycle gas line (9) or removed from the reactor system (20). The liquid phase is completely drawn off from the reactor system and sent to workup (12).

FIG. 2 shows a preferred embodiment of a mixing nozzle (3).

At the bottom, liquid enters the mixing nozzle (13) and is first set into rotation by means of a swirl body (15) and then, with a speed of preferably from 10 to 70 m/s in the first nozzle (16), enters the second chamber (17). In this chamber, cycle gas (14) is sucked in by the liquid jet, compressed and mixed intensively with the liquid. The gas-liquid mixture enters the reactor through a second nozzle, which sucks in liquid from the reactor (18), and is conveyed into the expansion tube (19) together with the gas-liquid mixture and finally into the guide tube (2) of the reactor.

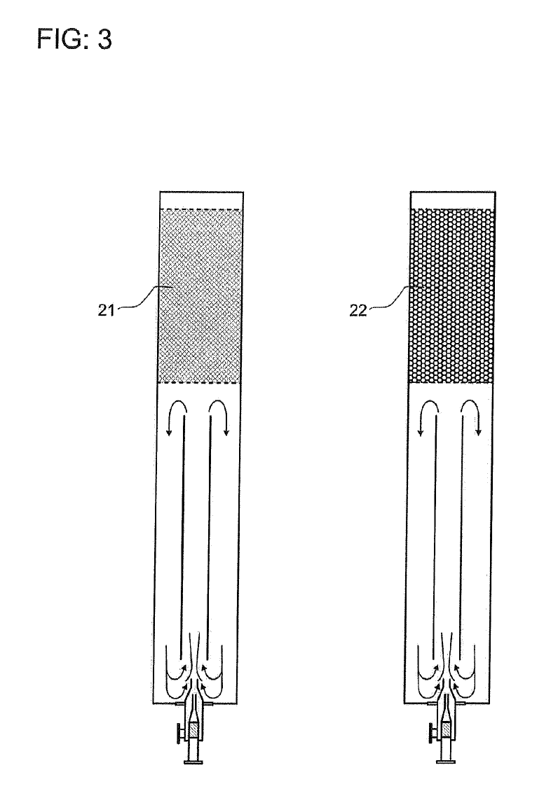

FIG. 3 shows two further embodiments in which it is shown how backmixing in vertical direction can be restricted in the bubble column reactor above the loop reactor. Instead of the perforated plates shown in FIG. 1, the entire volume of the bubble column reactor can be filled with structured packing (21) or with random packings (22).

The present invention constitutes a technically simple process for hydrogenating organic compounds, with which even highly exothermic hydrogenations can be performed within a narrow temperature range and with a very high end conversion in continuous mode. The process according to the invention makes it possible to perform the reaction within a defined temperature range in order thus to control the isomer ratio of the reactants and to prevent the formation of undesired by-products. The process enables a high conversion, and so the removal of unconverted reactants and reactants formed is simplified. In addition, the reaction regime and the apparatus configuration of the process can be implemented in a technically simple and economic manner.

It is evident from the working example shown in FIG. 1 that, in particular, the inventive hydrogenation reactor (1) does not need any technically complicated equipment at all, for example stirrers. By virtue of the simple design, this type of reactor can be used with particular preference, especially for reactions at high pressure. Moreover, this process enables long service lives, since the number of undesired shutdowns of the reactor to exchange or to regenerate the catalyst can be reduced, since fresh catalyst can be added to the process on reduction in activity or selectivity and spent catalyst can be discharged from the process.

The process according to the invention is illustrated in detail by the examples which follow.

### EXAMPLE 1

The example describes a continuous hydrogenation by the process according to the invention.

The preparation of the catalyst used, ruthenium(IV) oxide hydrate, was carried out according to Example 1 in DE 21 32 547, with the only exception that the final drying under reduced pressure was dispensed with. To prepare the catalyst suspension, 5500 g of the filtercake thus prepared (comprising 579 g of Ru calculated as 100% elemental ruthenium) were introduced into a stirred vessel and mixed intensively with 150 kg of a liquid product mixture (effluent of the hydrogenation reactor), such that the suspension obtained comprised 0.37% by weight of Ru.

The feedstock used for the hydrogenation was a melt of bis(4-aminophenyl)methane (4,4′-MDA) with a purity of approx. 99%. The reactant was used as a melt (melting point approx. 90° C.) without further dilution by a solvent.

The hydrogenation was performed in a continuous production plant. The reactor consisted of an upright, cylindrical high-pressure tube of diameter 600 mm and total height 16 m. In the upper part of the reactor were mounted four perforated plates at a distance of in each case 2 m. The perforated plates each had 36 holes of diameter 10 mm. The uppermost tray was present 2 m below the upper reactor lid. In the lower part of the reactor was centrally installed a guide tube with a length of 6.3 m and a diameter of 0.35 m, the upper edge of this tube having been mounted 1 m below the lowermost perforated plate. At the bottom of the reactor was installed centrally a multistage nozzle according to FIG. 2. The first nozzle had a diameter of 15 mm; the second nozzle of 33 mm. The expansion tube had a diameter of 40 mm at the narrowest point, 80 mm at the expansion tube exit, and the expansion tube possessed a length of 715 mm, measured from the tip of the second tube. The expansion tube projected into the guide tube by approx. 550 mm.

In the course of performance of the hydrogenation, a centrifugal pump is used to draw off approx. 25 m³/h of liquid from the lowermost part of the reactor, and the liquid is cooled by means of a heat exchanger and, after adding pure hydrogen, liquid reactant and catalyst solution, fed to the nozzle (connection 13). The first nozzle (16) generated a reduced pressure and thus drew in gas from the separator (11). The amount of cycle gas sucked in was approx. 5000 m³ (STP)/h.

500 kg/h of a melt of 4,4′-MDA with a temperature of 120° C. and 6.7 kg/h of the catalyst suspension were fed continuously to the reactor. The pressure in the reactor was regulated to 200 bar by flushing pure hydrogen into the pumped circulation system. The temperature in the reactor was 230° C. at the bottom of the reactor, 233° C. below the lowermost perforated plate and 238° C. at the uppermost point in the reactor. The gas-liquid mixture leaving at the top was separated in a separator. The liquid obtained was drawn off under level control, decompressed and cooled to 80° C. The gas obtained in the separator was fed completely back to the mixing nozzle and to the lower part of the reactor in an amount of 5000 m³ (STP)/h.

The analysis of the liquid product mixture by means of gas chromatography gave the following values (concentration data in % by weight):

| | Analysis of reaction |
|---|---|
| | effluent |
| | % by weight |
| Low-boiling by-products | 6.85 |
| High-boiling by-products | 3 |
| End product | 90 |
| Intermediate | 0.1 |
| Starting material | 0.05 |

End product: bis(4-aminocyclohexyl)methane
Intermediate: (4-aminocyclohexyl)(4-aminophenyl)methane
Starting material: bis(4-aminophenyl)methane

The conversion of the feedstock was 99.95% with a selectivity for the end product of approx. 90%.

### EXAMPLE 2

The reactor system used was that described in Example 1.

The feedstock used for the hydrogenation was a melt of bis(4-amino-3-methylphenyl)methane (“o-toluidine base”) having a purity of approx. 99%. The reactant was used as a melt (melting point approx. 160° C.) without further dilution by a solvent.

The catalyst used was ruthenium(IV) oxide hydrate, prepared according to Example 1 in DE 21 32 547. The catalyst was again suspended in liquid reactor effluent, so as to give rise to an Ru concentration of 0.37% by weight in this suspension.

750 kg/h of a melt of o-toluidine base were fed continuously to the reactor at a temperature of 180° C., as were 10 kg/h of the catalyst suspension. The pressure in the reactor was regulated to 200 bar by replenishing pure hydrogen into the pumped circulation system. The temperature in the reactor was 240° C. at the bottom of the reactor, 245° C. below the lowermost perforated plate and 248° C. at the uppermost point in the reactor. The gas-liquid mixture leaving at the top was separated in a separator. The liquid obtained was drawn off under level control, decompressed and cooled to 80° C. The gas obtained in the separator was completely fed back to the mixing nozzle and to the lower part of the reactor in an amount of 5000 m³ (STP)/h.

The analysis of the liquid product mixture by means of gas chromatography gave the following values (concentration data in % by weight):

| | Analysis of reaction |
|---|---|
| | effluent |
| | % by weight |
| Low-boiling by-products | 3.5 |
| High-boiling by-products | 1.3 |
| End product | 95.1 |
| Intermediate | 0.08 |
| Starting material | 0.02 |

End product: bis(4-amino-3-methylcyclohexyl)methane
Intermediate: (4-amino-3-methylcyclohexyl)(4-amino-3-methylphenyl)methane
Starting material: bis(4-amino-3-methylphenyl)methane

The conversion of the feedstock was >99.9% with a selectivity for the end product of approx. 95%.

1-20. (canceled)

21. A continuous process for hydrogenating organic compounds in a polyphasic system in the presence of a homogeneous or heterogeneous catalyst, which comprises performing the process in two stages, the first stage being performed in a loop reactor with an external heat exchanger and the second stage in a bubble column reactor with limited backmixing.

22. The process according to claim 21, wherein the hydrogenation is performed in the presence of a heterogeneous catalyst.

23. The process according to claim 21, wherein the organic compounds used are aromatic compounds.

24. The process according to claim 23, wherein the aromatic compounds used are polymeric MDA, aniline, 2,4-diaminotoluene, 2,6-diaminotoluene, o-phenylenediamine, m-phenylenediamine, p-phenylenediamine, bis(4-aminophenyl)methane (MDA), meta-xylenediamine (MXDA), bis(4-amino-3-methylphenyl)methane and/or bis(4-amino-3,5-dimethylphenyl)methane.

25. The process according to claim 21, wherein the first stage of the process is performed in a loop reactor with internal loop flow.

26. The process according to claim 21, wherein the backmixing in the bubble column reactor of the second stage is restricted by internals.

27. The process according to claim 26, wherein the internals are gas- and liquid-permeable sparging trays.

28. The process according claim 26, wherein the bubble column reactor is divided into several individual segments by the sparging trays and the number of individual segments is from 1 to 20.

29. The process according to claim 26, wherein the internals are random packings or structured packings.

30. The process according to claim 21, wherein the two stages of the process are arranged in one apparatus.

31. The process according to claim 21, wherein a maximum temperature difference of 20 K occurs between the reactant feed in the loop reactor of the first process stage and the exit of the gas-liquid mixture in the bubble column reactor of the second process stage.

32. The process according to claim 21, wherein the conversion in the loop reactor is from 92 to 98%.

33. A hydrogenation reactor (1) in a tall cylindrical design comprising a concentric guide tube (2) arranged in the lower reaction region and a mixing nozzle (3) directed upward, through which reactants and reaction mixture can be supplied, a pump (4) and a heat exchanger (5), which are present in an external pump circulation system which leads from the reactor to the mixing pump, and with one or more gas- and liquid-permeable internals (10) mounted in the upper region of the reactor, where the ratio of total length to diameter of the hydrogenation reactor (1) is in the range from 5:1 to 100:1 and the ratio of the volume of the upper region of the reactor to the volume of the lower region of the reactor is from 0.05:1 to 10:1.

34. The hydrogenation reactor (1) according to claim 33, wherein feeds (6, 7 and 8) for reactants are provided in the external pumped circulation system.

35. The hydrogenation reactor (1) according to claims 33, wherein the gas- and liquid-permeable internals (10) are sparging trays, such as perforated plates.

36. The hydrogenation reactor (1) according to claim 35, wherein the volume of a segment divided by the sparging trays is in the range from 5 to 50%, based on the total volume of the reactor, and the number of individual segments is from 1 to 20.

37. The hydrogenation reactor (1) according to claim 36, wherein the ratio of the volume of the upper region of the reactor to the volume of the lower region of the reactor is in the range from 0.4:1 to 1.5:1, and the number of individual segments is from 2 to 6.

38. The hydrogenation reactor (1) according to claims 33, wherein an exit point for the reaction mixture is present in the upper region of the reactor, which is separated in a separator (11) into a liquid stream/product stream (12) and a gas stream.

39. A process for hydrogenating organic compounds in a polyphasic system in the presence of a homogeneous or heterogeneous catalyst, which comprises performing the reaction in a reactor according to claim 33.
